(19) United States
(12) Patent Application Publication
(10) Pub. No.: US 2011/0160264 A1
(43) Pub. Date: Jun. 30, 2011

(54) ORALLY ADMINISTRABLE FILM DOSAGE FORMS CONTAINING ONDANSETRON

(75) Inventors: Garry L. Myers, Kingsport, TN (US); Madhu Hariharan, Munster, IN (US)

(73) Assignee: MONOSOL RX, LLC, Warren, NJ (US)

(21) Appl. No.: 12/978,847

(22) Filed: Dec. 27, 2010

Related U.S. Application Data
(60) Provisional application No. 61/290,376, filed on Dec. 28, 2009.

(51) Int. Cl. A61K 31/4178; A61P 1/08

(52) U.S. Cl. 514/397

(57) ABSTRACT

The invention relates to orally administrable, disintegrating film dosage forms which include ondansetron and methods of orally administering the film dosage forms.

### CROSS-REFERENCE TO RELATED APPLICATIONS

This application claims priority to U.S. Provisional Application No. 61/290,376, filed Dec. 28, 2009, the entire contents of which are incorporated herein by reference.

### FIELD OF THE INVENTION

The invention relates to orally administrable, disintegrating film dosage forms which include ondansetron and methods of orally administering the film dosage forms.

### BACKGROUND OF THE INVENTION

Ondansetron is a selective antagonist of 5-hydroxytryptamine (5HT, or serotonin) at 5-HT₃ receptors. Ondansetron is also referred to as 9-methyl-3-[(2-methyl-1H-imidazol-1-yl)methyl]-1,2,3,9-tetrahydro-4H-carbazol-4-one and has the following structural formula:

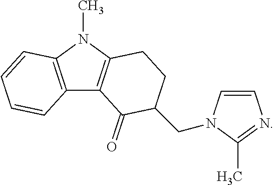

Ondansetron has been described in U.S. Pat. No. 4,695,578 and U.S. Pat. No. 4,753,789, which are incorporated by reference in their entirety.

Ondansetron is highly effective for the treatment and prevention of nausea and/or vomiting. Currently, ondansetron is administered as an oral tablet, an orally disintegrating tablet, an oral solution, and an injectable. However, these routes of administration may not be suitable or convenient for all patients. For example, some patients have difficulty swallowing and/or some patients expel the drug after administration. There is an unmet need in the art for an oral dosage form which include ondansetron which can be administered conveniently without requiring water for ingestion, which can disintegrate rapidly, and which has a decreased potential for a patient to expel the dosage form after administration and placement of the dosage form in the oral cavity.

U.S. Patent Application Publication No. 2005/0037055, to Yang et al. discloses rapid-dissolve film products containing at least one water-soluble polymer, which demonstrate a non-self-aggregating uniform heterogeneity. U.S. Patent Application Publication No. 2005/0037055 discloses that the rapid-dissolve film products can comprise a variety of different active ingredients, including anti-nausea agents.

U.S. Patent Application Publication No. 2007/0122455, to Myers et al. discloses water-soluble films incorporating anti-tacking agents and methods of their preparation. U.S. Patent Application Publication No. 2007/0122455 discloses that the water-soluble films can comprise a variety of different active ingredients, including anti-nausea agents.

U.S. Patent Application Publication No. 2007/0149731, to Myers, discloses pH-modulated films containing at least one component having a non-neutral pH when combined with water, and methods of their preparation. U.S. Patent Application Publication No. 2007/0149731 discloses that the pH-modulated films can comprise a variety of different active ingredients, including granisetron.

U.S. Patent Application Publication No. 2008/0050422, to Myers et al., discloses a fast-dissolving film product containing at least one drug and a water-soluble polymer. U.S. Patent Application Publication No. 2008/0050422 discloses that the fast-dissolving film product can comprise a variety of different active ingredients, including granisetron.

U.S. Patent Application Publication No. 2008/0075825, to Fuisz et al. discloses an edible water-soluble film containing at least one water-soluble polymer and a foam reducing flavoring agent. U.S. Patent Application Publication No. 2008/0075825 discloses that the edible water-soluble film can comprise a variety of different active ingredients, including granisetron.

The present invention addresses the unmet needs in the art and provides a disintegrating film dosage form which includes ondansetron and which is convenient and easy to administer to subjects, which disintegrates rapidly, and which improves patient compliance.

All references cited herein are hereby incorporated by reference in their entirety.

### SUMMARY OF THE INVENTION

The present invention provides an orally administrable, disintegrating film dosage form comprising ondansetron, wherein the dosage form provides a mean maximum plasma concentration (Cₘₐₓ) of about 2.0 to about 4.5 μg/L per mg (microgram per liter per milligram) of ondansetron in the dosage form after oral administration of a single dosage form to human subjects in a fed state. The present invention also provides an orally administrable, disintegrating film dosage form comprising ondansetron, wherein the dosage form provides a mean maximum plasma concentration (Cₘₐₓ) of about 3.0 to about 6.9 μg/L per mg of ondansetron in the dosage form after oral administration of a single dosage form to human subjects in a fasted state.

The present invention also provides an orally administrable, disintegrating film dosage form including ondansetron, wherein the dosage form provides a mean plasma concentration over 0-24 hours (AUC₀₋₂₄) of about 11.6 to about 36.0 μg·hr/L per mg of ondansetron in the dosage form after oral administration of a single dosage form to human subjects in a fed state. The present invention also provides an orally administrable, disintegrating film dosage form including ondansetron, wherein the dosage form provides a mean plasma concentration over 0-24 hours (AUC₀₋₂₄) of about 19.4 to about 44.0 μg·hr/L per mg of ondansetron in the dosage form after oral administration of a single dosage form to human subjects in a fasted state.

The present invention also provides an orally administrable, disintegrating film dosage form including ondansetron, wherein the dosage form provides a time to reach maximum plasma concentration (Tₘₐₓ) of ondansetron of less than about 4 hours after oral administration of a single dosage form to human subjects in a fed state. The present invention also provides an orally administrable, disintegrating film dosage form including ondansetron, wherein the dosage form provides a time to reach maximum plasma concentration (Tₘₐₓ) of ondansetron of less than about 3 hours after oral administration of a single dosage form to human subjects in a fasted state.

The present invention also provides an orally administrable, disintegrating film dosage form including ondansetron, wherein the dosage form is configured such that when the dosage form is administered to human subjects in a fasted state with sequential administration of water, the mean maximum plasma concentration (Cₘₐₓ) of ondansetron achieved after administration of the dosage form is within about ±10% of the mean maximum plasma concentration (Cₘₐₓ) of ondansetron achieved after administration of the dosage form when administered to human subjects in a fasted state without sequential administration of water.

The present invention also provides an orally administrable, disintegrating film dosage form including ondansetron, wherein the dosage form is configured such that when the dosage form is administered to human subjects in a fasted state with administration of water, the mean plasma concentration over 0-24 hours (AUC₀₋₂₄) of ondansetron achieved after administration of the dosage form is within about ±10% of the mean plasma concentration over 0-24 hours (AUC₀₋₂₄) of ondansetron achieved after administration of the dosage form when administered to human subjects in a fasted state without sequential administration of water.

The present invention also provides an orally administrable, disintegrating film dosage form including ondansetron, wherein the dosage form is configured such that when the dosage form is administered to human subjects in a fasted state with sequential administration of water, the time to reach maximum plasma concentration (Tₘₐₓ) of ondansetron achieved after administration of the dosage form is within about ±20% of the time to reach maximum plasma concentration (Tₘₐₓ) of ondansetron achieved after administration of the dosage form when administered to human subjects in a fasted state without administration of water.

The present invention also provides methods of orally administering a disintegrating film dosage form including ondansetron to human subjects for the treatment and prevention of nausea and/or vomiting associated with any condition which causes nausea, including, for example, gastritis, motion sickness, cancer chemotherapy, radiotherapy, and surgery.

### DETAILED DESCRIPTION OF THE INVENTION

The present invention provides an orally administrable, disintegrating film dosage form including ondansetron. The term “ondansetron” refers to ondansetron, pharmaceutically acceptable salts, hydrates, solvates, polymorphs, complexes, and pro-drugs thereof. The term “ondansetron” may refer to the racemic mixture or enantiomers of ondansetron. The term “ondansetron” further includes any moiety which yields the ondansetron active component. In preferred embodiments, “ondansetron” is the hydrochloride salt of ondansetron or the base of ondansetron. As used herein, the term “complex” is intended to include any construct including ondansetron and a ligand to which it may be associated by any association, including by ionic bond, by covalent bond, by inclusion, or by any other methods of forming a complex desired.

The compositions of the present invention provide the Cₘₐₓ and AUC values as recited herein regardless of whether the composition was administered to a patient in the fed or fasted state, with or without water.

As used herein, the terms “disintegrate”, “disintegrating”, and “disintegrated” includes dissolving, dispersing, or otherwise breaking apart for release of the drug particles and other components contained therein, such that they may be swallowed and/or absorbed into the body, including absorption into the oral cavity and/or the gastrointestinal tract.

It will be understood that the term “film” includes delivery systems of any thickness, including films, sheets, discs, wafers, and the like, in any shape, including rectangular, square, or other desired shape. The film may be in the form of a continuous roll of film or may be sized to a desired length and width. The films described herein may be any desired thickness and size suitable for the intended use. For example, a film of the present invention may be sized such that it may be placed into the oral cavity of the user. Other films may be sized for application to the skin of the user, i.e., a topical use. For example, some films may have a relatively thin thickness of from about 0.1 to about 10 mils, while others may have a somewhat thicker thickness of from about 10 to about 30 mils. For some films, especially those intended for topical use, the thickness may be even larger, i.e., greater than about 30 mils. In addition, the term “film” includes single-layer compositions as well as multi-layer compositions, such as laminated films, coatings on films and the like. The composition in its dried film form maintains a uniform distribution of components through the application of controlled drying of the film. Films may include a pouch or region of ondansetron between two films.

The ondansetron may be dispersed throughout the film, or it may be deposited onto one or more surfaces of the film. In either way, the amount of ondansetron per unit area is desirably uniform throughout the film. It is desired that the films of the present invention include a uniformity of component distribution throughout the volume of a given film. Such uniformity includes a substantially uniform amount of ondansetron per unit volume of the film, whether the ondansetron is within the matrix of the film or coated, laminated, or stabilized on one or more surfaces thereof. When such films are cut into individual units, the amount of the agent in the unit can be known with a great deal of accuracy.

Uniformity of ondansetron throughout the film is important in administering an accurate and effective dose of ondansetron to a user. Various methods of forming uniform films, as well as various additives and fillers, may be used, including those methods and materials described in U.S. Pat. Nos. 7,425,292 and 7,357,891 and U.S. Publication No. 2005/0037055, which are herein incorporated by reference in their entireties.

The term “disintegrating film dosage form” refers to a dosage form in the form of a sheet or film that can be administered orally to a subject, preferably a human subject. The disintegrating film dosage form contains ondansetron and one or more pharmaceutically acceptable excipients. The ondansetron in the film dosage form may be disintegrated in solution or suspended in the film. Upon placement of the film dosage form in the mouth of a subject, the film dosage form disintegrates, releasing the ondansetron, and making it available for absorption in the oral cavity or gastrointestinal tract. In preferred embodiments, the disintegrating film dosage form rapidly disintegrates, meaning that substantially all of the film dosage form disintegrates in the oral cavity in less than 5 minutes, more preferably in less than 3 minutes, and most preferably in less than 1 minute, after placement in the oral cavity. “Substantially all of the film dosage form” refers to more than about 50%, preferably more than about 75%, and more preferably more than about 90% of the film dosage form.

Examples of suitable disintegrating film dosage forms are described in U.S. Pat. No. 7,425,292; U.S. Pat. No. 7,357,891; U.S. Patent Application Publication No. 2006/0147493; U.S. Patent Application Publication No. 2005/0037055; U.S. Patent Application Publication No. 2006/0039958; U.S. Patent Application Publication No. 2007/0122455; U.S. Patent Application Publication No. 2008/0050422; U.S. Patent Application Publication No. 2007/0149731; U.S. Patent Application Publication No. 2007/0281003; U.S. Patent Application Publication No. 2008/0044454; and U.S. Patent Application Publication No. 2008/0075825, which are each incorporated by reference in their entirety.

One embodiment of the present invention provides an orally administrable, disintegrating film dosage form including ondansetron, wherein the dosage form provides a mean maximum plasma concentration (Cₘₐₓ) of about 2.0 to about 4.5 μg/L, preferably about 2.2 to about 4.4 μg/L, and more preferably about 2.3 to about 4.3 μg/L, per mg of ondansetron in the dosage form, after oral administration of a single dosage form to human subjects in a fed state. The present invention also provides an orally administrable, disintegrating film dosage form including ondansetron, wherein the dosage form provides a mean maximum plasma concentration (Cₘₐₓ) of about 3.0 to about 6.9 μg/L, preferably about 3.2 to about 6.7 μg/L, and more preferably about 3.3 to about 6.5 μg/L, per mg of ondansetron in the dosage form after oral administration of a single dosage form to human subjects in a fasted state. The “mean maximum plasma concentration (Cₘₐₓ)” refers to the maximum blood plasma concentration of the drug substance and/or active metabolites.

A human subject is in the “fed state” when the dosage form is administered within about 2 hours, preferably about 1 hour, more preferably about 30 minutes, after consuming a meal. Preferably the meal is high in fat. A human is in the “fasted state” when the dosage form is administered no earlier than at least 10 hours, preferably at least 12 hours, and more preferably at least 14 hours after consuming a meal.

The present invention also provides an orally administrable, disintegrating film dosage form including ondansetron, wherein the dosage form provides a mean plasma concentration over 0-24 hours (AUC₀₋₂₄) of about 11.6 to about 36.0 μg·hr/L, preferably about 12.9 to about 34.8 μg·hr/L, and more preferably 14.1 to about 33.5 μg·hr/L, per mg of ondansetron in the dosage form after oral administration of a single dosage form to human subjects in a fed state. The present invention also provides an orally administrable, disintegrating film dosage form including ondansetron, wherein the dosage form provides a mean plasma concentration over 0-24 hours (AUC₀₋₂₄) of about 19.4 to about 44.0 μg·hr/L, preferably about 20.8 to about 42.7 μg·hr/L, and more preferably about 22.0 to about 41.5 μg·hr/L, per mg of ondansetron in the dosage form after oral administration of a single dosage form to human subjects in a fasted state. The “mean plasma concentration over 0-24 hours (AUC₀₋₂₄)” refers to the area under the plasma concentration curve over 0 to 24 hours after administration.

The present invention also provides an orally administrable, disintegrating film dosage form including ondansetron, wherein the dosage form provides a time to reach maximum plasma concentration (Tₘₐₓ) of ondansetron of less than about 4 hours, preferably less than about 3 hours, and more preferably about 2½ to 3 hours, after oral administration of a single dosage form to human subjects in a fed state. The present invention also provides an orally administrable, disintegrating film dosage form including ondansetron, wherein the dosage form provides a time to reach maximum plasma concentration (Tₘₐₓ) of ondansetron of less than about 3 hours, preferably less than about 2 hours, more preferably less than about 1½ hours, and most preferably about 1 to 1½ hours, after oral administration of a single dosage form to human subjects in a fasted state. The time to reach maximum plasma concentration (Tₘₐₓ) refers to the time to reach mean maximum plasma concentration (Cₘₐₓ).

Preferably the mean maximum plasma concentration (Cₘₐₓ), the mean area under the plasma concentration time curve over 0-24 hours (AUC₀₋₂₄), and time to reach maximum plasma concentration (Tₘₐₓ) are measured after administration of disintegrating film dosage forms including about 4 mg or more of ondansetron, preferably including about 8 mg of ondansetron. Unless otherwise specified, preferably the mean maximum plasma concentration (Cₘₐₓ), mean plasma concentration over 0-24 hours (AUC₀₋₂₄), and time to reach maximum plasma concentration (Tₘₐₓ) are measured after the film dosage form is administered with sequential administration of water. The film dosage form is administered to a human subject “with sequential administration of water” if the human subject swallows about 240 mL of water, preferably room temperature drinking water, after the film dosage form is orally administered, allowed to disintegrate, and swallowed with saliva.

The present invention also provides an orally administrable, disintegrating film dosage form including ondansetron, wherein the dosage form is configured such that when the dosage form is administered to human subjects in a fasted state with sequential administration of water, the mean maximum plasma concentration (Cₘₐₓ) of ondansetron achieved after administration of the dosage form is within about ±10%, preferably within about ±8%, and more preferably within about ±5%, of the mean maximum plasma concentration (Cₘₐₓ) of ondansetron achieved after administration of the dosage form when administered to human subjects in a fasted state without administration of water. For the purposes of this invention, the film dosage form is administered to a human subject “without administration of water” if the human subject does not consume water within about one hour before or after the film dosage form is orally administered, allowed to disintegrate, and swallowed with saliva.

The present invention also provides an orally administrable, disintegrating film dosage form including ondansetron, wherein the dosage form is configured such that when the dosage form is administered to human subjects in a fasted state with sequential administration of water, the mean plasma concentration over 0-24 hours (AUC₀₋₂₄) of ondansetron achieved after administration of the dosage form is within about ±10%, preferably within about ±5%, more preferably within about ±2%, of the mean plasma concentration over 0-24 hours (AUC₀₋₂₄) of ondansetron achieved after administration of the dosage form when administered to human subjects in a fasted state without administration of water.

The present invention also provides an orally administrable, disintegrating film dosage form including ondansetron, wherein the dosage form is configured such that when the dosage form is administered to human subjects in a fasted state with sequential administration of water, the time to reach maximum plasma concentration (Tₘₐₓ) of ondansetron achieved after administration of the dosage form is within about ±20%, preferably within about ±18%, and more preferably within about ±15% of the time to reach maximum plasma concentration (Tₘₐₓ) of ondansetron achieved after administration of the dosage form when administered to human subjects in a fasted state without administration of water.

Preferably the mean maximum plasma concentration (Cₘₐₓ), mean plasma concentration over 0-24 hours (AUC₀₋₂₄), and time to reach maximum plasma concentration (Tₘₐₓ) are measured after administration of disintegrating film dosage forms including about 4 mg or more of ondansetron, preferably including about 8 mg of ondansetron.

The ondansetron used in the present invention may be in particulate form. The ondansetron may be any particle size desired. The ondansetron in the film may include smaller sized particles, intermediate sized particles, larger sized particles, and combinations thereof. For smaller sized particles, the ondansetron may have a particle size of about 0.5 to about 10.0 microns in diameter, and more specifically between about 0.5 and about 1.5 microns in diameter. In some embodiments, about 10 percent of the particles in the film may have a size less than about 0.5 to about 10.0 microns in diameter, and more specifically between about 0.5 and about 1.5 microns in diameter.

For intermediate sized particles, the ondansetron may have a particle size of about 1.0 to about 50.0 microns in diameter, and more specifically between about 2.0 to about 6.0 microns in diameter. In some embodiments, about 50 percent of the particles in the film may have a size less than about 1.0 to about 50.0 microns in diameter, and more specifically between about 2.0 to about 6.0 microns in diameter.

For larger sized particles, the ondansetron may have a particle size of about 3.0 to about 200.0 microns in diameter, and more specifically between about 7.0 to about 25.0 microns in diameter. In some embodiments, about 90 percent of the particles in the film may have a size less than about 3.0 to about 200.0 microns in diameter, and more specifically between about 7.0 to about 25.0 microns in diameter.

The disintegrating film dosage form includes one or more film-forming polymers. The film-forming polymer may be a water soluble polymer, a water insoluble polymer, or a combination of one or more water soluble polymers and/or water insoluble polymers.

As used herein the phrase “water soluble polymer” and variants thereof refer to a polymer that is at least partially soluble in water, and desirably fully or predominantly soluble in water, or absorbs water. Polymers that absorb water are often also referred to as being water swellable polymers, and this term is synonymous for the purposes of the present invention. The materials useful with the present invention may be water soluble at room temperature and other temperatures, such as temperatures exceeding room temperature. Moreover, the materials may be water soluble at pressures less than atmospheric pressure. Desirably, the water soluble polymers have at least 20 percent by weight water uptake. Water soluble polymers having a 25 or greater percent by weight water uptake are also useful. Films or dosage forms of the present invention formed from such water soluble polymers are desirably sufficiently water soluble to be disintegratable upon contact with bodily fluids.

Examples of water soluble polymers include, but are not limited to water-soluble polysaccharides, cellulose polymers or cellulosic derivative polymers, and water-soluble synthetic polymers.

Water soluble polysaccharides include, but are not limited to alginates such as sodium alginate, carrageenans, guar gum, acacia gum, agar, xanthan gum, gellan gum, arabic gum and related gums (gum ghatti, gum karaya, gum tragancanth), and pectin.

Examples of cellulosic polymers and cellulosic derivative polymers include, but are not limited to alkylcelluloses, hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate; carboxyalkylcelluloses, carboxyalkylalkylcelluloses, carboxyalkylcellulose esters such as carboxymethylcellulose and their alkali metal salts. In some preferred embodiments, the cellulose polymer and cellulosic derivative polymers include, but are not limited to, methylcellulose, ethylcellulose, hydroxypropyl ethylcellulose, hydroxyethyl cellulose, hydroxypropyl methylcellulose, cellulose acetate phthalate, hydroxypropyl methyl cellulose phthalate, and combinations thereof. The most preferred cellulose polymer is hydroxypropyl methylcellulose. In preferred embodiments, the disintegrating film dosage form includes one or more cellulose polymers or cellulosic derivative polymers.

Synthetic polymers include, but are not limited to polyacrylic acids and polyacrylic acid esters, polymethacrylic acids and polymethacrylic acid esters, polyalkylene oxides, such as polyethylene oxide, polyvinylacetates, polyvinylalcohols, polyvinylacetatephthalates (PVAP), polyvinylpyrrolidone (PVP), polyvinyl acetate copolymers, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quarternized if desired. The most preferred synthetic polymer is polyethylene oxide. In preferred embodiments, the disintegrating film dosage form comprises one or more water-soluble synthetic polymers.

The disintegrating film dosage form of the invention also may include a variety of other pharmaceutically acceptable excipients. These may include, without limitation, surfactants; plasticizers which assist in compatibilizing the components within the mixture; polyalcohols; anti-foaming agents, such as silicone-containing compounds, which promote a smoother film surface by releasing gases, such as oxygen, from the film; and thermo-setting gels such as pectin, carageenan, and gelatin, which help in maintaining the dispersion of components.

The variety of pharmaceutically acceptable excipients that can be incorporated into the inventive compositions may provide a variety of different functions. Examples of classes of pharmaceutically acceptable excipients include excipients, lubricants, buffering agents, stabilizers, blowing agents, pigments, coloring agents, fillers, bulking agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Additional useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins.

Further pharmaceutically acceptable excipients may be inorganic materials, such as the oxides of magnesium aluminum, silicon, titanium, etc. desirably in a concentration range of about 0.02% to about 3% by weight and desirably about 0.02% to about 1% based on the weight of all components.

Further examples of pharmaceutically acceptable excipients are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, which, if present, may be added in concentrations ranging from about 0.5% to about 10% by weight of the dosage form.

In preferred embodiments, the disintegrating film dosage form includes one or more film-forming polymers, preferably one or more cellulose polymers or cellulosic derivative polymers. In preferred embodiments, the total amount of the one or more film-forming polymers ranges from about 10% to about 70%, preferably about 20% to about 60%, and more preferably about 30% to about 50%, by weight of the dosage form. In some preferred embodiments, the dosage form includes two or more film-forming polymers, preferably at least one cellulose polymer or cellulosic derivative polymer and at least one synthetic polymer.

In some embodiments wherein the dosage form includes at least one cellulose polymer or cellulosic derivative polymer and at least one synthetic polymer, the cellulose polymers or cellulosic derivative polymers and the synthetic polymers are present in a weight ratio ranging from about 10:1 to about 1:10, more preferably about 7:1 to about 1:7, and most preferably about 4:1 to about 1:4. In some preferred embodiments, the amount of the cellulose polymers or cellulosic derivative polymers is greater than the amount of the synthetic polymers, and the weight ratio preferably ranges from about 1:1 to about 3:1.

There may be further added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂, C₁₄, C₁₆, C₁₈, C₂₀ and C₂₂ fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂, C₁₄, C₁₆, C₁₈, C₂₀ and C₂₂ fatty acids. The total amounts used of the fats, mono-, di-glycerides and/or lecithins are up to about 5% and preferably within the range of about 0.5% to about 2% by weight of the total composition.

Lecithin or other surface active agents may be used in the present invention. The surface active agents, if present, may be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

Other components include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

Anti-foaming and/or de-foaming components may also be used with the films of the present invention. These components aid in the removal of air, such as entrapped air, from the film-forming compositions, which may lead to non-uniform films. Simethicone and silicone-containing compounds, such as silicone dioxide, are useful anti-foaming and/or de-foaming agents. Flavoring agents may be used as de-foaming agents, as described in U.S. Patent Publication No. 2008/0075825, the entire contents of which are incorporated by reference herein in their entirety. The present invention, however, is not so limited and other anti-foam and/or de-foaming agents may suitable be used.

Buffering agents or pH adjusting agents may also be used, such as calcium carbonate, sodium bicarbonate, citric acid, tartaric acid, succinic acid, maleic acid, and fumaric acid.

Antioxidants and preservatives may also be added to the film. Examples of antioxidants and preservatives include, but are not limited to parabens, such as methyl paraben, ethyl paraben, propyl paraben, and butyl paraben, benzoic acid, sodium benzoate, sorbic acid, sodium sorbate, cetrimide, benzalkonium chlorise, cetylpyridium chloride, benzaethonium chloride, phenylmercuric nitrate, benzyl alcohol, phenylethyl alcohol, bronabol, chlorbutanol, chlorhexidine, butylated hydroxyanisole, butylated hydroxytoluene, tert-butyl hydroquinone, and 4-hydroxymethyl-2,6,-di-ter-butylphenol.

Color additives can be used in preparing the films. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and erythritol. Also contemplated are hydrogenated starch hydrolysates and the synthetic sweetener 3,6-dihydro-6-methyl-1-1-1,2,3-o-xathiazin-4-one-2,2-dioxide, particularly the potassium salt (acesulfame-K), ammoniated glycyrrhizin and monoammonium glycyrrhizinate, and sodium and calcium salts thereof, and natural intensive sweeteners. Other sweeteners may also be used.

The present invention also provides for methods of using the disintegrating film dosage forms. In preferred embodiments, the disintegrating film dosage form is placed in the oral cavity of the subject, such as on the tongue, and allowed to disintegrate completely. The disintegrating film dosage form may be administered to a subject in a fed state or a fasted state. The disintegrating film dosage form may also be administered with or without the administration of water. In some embodiments, more than one disintegrating film dosage form may be administered sequentially within a single dosage administration. When more than one disintegrating film dosage form is administered sequentially, preferably the subject places the disintegrating film dosage form in the oral cavity and allows the dosage form to completely disintegrate before administration of the next dosage form.

The disintegrating film dosage forms including ondansetron may be administered to any subject, adult or pediatric, for any use which benefits from the administration of ondansetron. For example, the disintegrating film dosage form of the present invention can be used to treat, prevent, or reduce the severity or occurrence of any symptom or condition associated with 5-HT₃ receptors and which would benefit from antagonism at the 5-HT₃ receptor. For example, the disintegrating film dosage forms of the present invention may be suitable to prevent, treat, or reduce the occurrence of nausea and/or vomiting.

The nausea and/or vomiting may be associated with chemotherapy that is emetogenic. “Emetogenic” chemotherapy is chemotherapy which results in symptoms of nausea and/or vomiting after administration. In preferred embodiments, the emetogenic chemotherapy is a highly emetogenic cancer chemotherapy or a moderately emetogenic cancer chemotherapy. “Highly emetogenic cancer chemotherapy” as used herein includes a chemotherapy where over 90% of patients experience some degree of nausea and/or vomiting. Examples of highly emetogenic cancer chemotherapies include, but are not limited to chemotherapies which involve administration of cisplatin in doses ≧50 mg/m². In embodiments wherein the disintegrating film dosage form is used for the prevention of nausea and/or vomiting associated with highly emetogenic cancer chemotherapies, the typical adult oral dosage is 24 mg of ondansetron, given successfully as three (3) disintegrating film dosage forms, each comprising 8 mg of ondansetron. In preferred embodiments, the disintegrating film dosage forms are administered about 5 to about 60 minutes, preferably about 15 to about 45 minutes, more preferably about 30 minutes before the start of a single day of highly emetogenic cancer chemotherapy.

In some preferred embodiments, the disintegrating film dosage forms may be administered to subjects receiving initial and repeat courses of moderately emetogenic cancer chemotherapy. “Moderately emetogenic chemotherapy” as used herein includes a chemotherapy where about 30% to about 90% of patients experience some degree of nausea and/or vomiting. Examples of highly emetogenic cancer chemotherapies include, but are not limited to chemotherapies which involve administration of cyclophosphamide-based chemotherapy containing methotrexate or doxorubicin.

In embodiments wherein the disintegrating film dosage form is used for the prevention of nausea and/or vomiting associated with moderately emetogenic cancer chemotherapies, the typical oral dosage for adults and children aged 12 years and older is one (1) disintegrating film dosage form comprising 8 mg of ondansetron, administered twice a day. In preferred embodiments, the first disintegrating film dosage form comprising 8 mg of ondansetron is administered about 5 to about 60 minutes, preferably about 15 to about 45 minutes, more preferably about 30 minutes before the start of the emetogenic chemotherapy, with the subsequent disintegrating film dosage form comprising 8 mg of ondansetron administered about 5 to 15 hours, preferably about 6 to about 10 hours, more preferably about 8 hours, after administration of the first dosage form. In preferred embodiments, one disintegrating film dosage form comprising 8 mg of ondansetron is administered twice a day, preferably every about 8 to about 16 hours, preferably every about 10 to about 14 hours, more preferably every about 12 hours, for about 1 to 5 days, preferably about 1 to 2 days after completion of chemotherapy.

In embodiments wherein the disintegrating film dosage form is used for the prevention of nausea and/or vomiting associated with moderately emetogenic cancer chemotherapies, the typical oral dosage for children under about 12 years of age, preferably about 4 to 11 years of age, is one (1) disintegrating film dosage form including 4 mg of ondansetron, administered three (3) times a day. In preferred embodiments, the first disintegrating film dosage form including 4 mg of ondansetron is administered about 5 to about 60 minutes, preferably about 15 to about 45 minutes, more preferably about 30 minutes, before the start of the emetogenic chemotherapy, with the subsequent disintegrating film dosage form including 4 mg of ondansetron administered: (1) about 2 to about 6 hours, preferably about 4 hours, and (2) about 6 to about 10 hours, preferably about 8 hours, after the administration of the first disintegrating film dosage form. In preferred embodiments, one disintegrating film dosage form including 4 mg of ondansetron is administered three times a day, preferably every about 4 to about 12 hours, preferably every about 6 to about 10 hours, more preferably every about 8 hours, for about 1 to 5 days, preferably about 1 to 2 days, after completion of chemotherapy.

The nausea and/or vomiting may also be associated with radiotherapy. The radiotherapy may include, but is not limited to, total body irradiation, single high-dose fraction radiotherapy to the abdomen, and daily fractionated radiotherapy to the abdomen. In embodiments wherein the disintegrating film dosage form is used for the prevention of nausea and/or vomiting associated with radiation, the typical oral dosage for adults is one (1) disintegrating film dosage form comprising 8 mg of ondansetron, administered three times a day.

In embodiments wherein the disintegrating film dosage form is used for the prevention of nausea and/or vomiting associated with total body irradiation, preferably one disintegrating film dosage form including ondansetron is administered about 15 minutes to about 4 hours, preferably about 30 minutes to about 3 hours, and more preferably about 1 to about 2 hours, before each fraction of radiotherapy administered each day.

In embodiments wherein the disintegrating film dosage form is used for the prevention of nausea and/or vomiting associated with single high-dose fraction radiotherapy to the abdomen, preferably one disintegrating film dosage form including ondansetron is administered about 15 minutes to about 4 hours, preferably about 30 minutes to about 3 hours, and more preferably about 1 to about 2 hours, before radiotherapy. Subsequent administrations of the disintegrating film dosage form including 8 mg of ondansetron can be administered every about 4 to about 12 hours, preferably every about 6 to about 10 hours, more preferably every about 8 hours, for about 1 to 5 days, preferably about 1 to 2 days, after completion of radiotherapy.

In embodiments wherein the disintegrating film dosage form is used for the prevention of nausea and/or vomiting associated with daily fractionated radiotherapy to the abdomen, preferably one disintegrating film dosage form including ondansetron is administered about 15 minutes to about 4 hours, preferably about 30 minutes to about 3 hours, and more preferably about 1 to about 2 hours, before radiotherapy. Subsequent administrations of the disintegrating film dosage form including 8 mg of ondansetron can be administered every about 4 to about 12 hours, preferably every about 6 to about 10 hours, more preferably every about 8 hours, for each day radiotherapy is given.

The nausea and/or vomiting may also be postoperative nausea and/or vomiting. In some embodiments, administration of the disintegrating film dosage form including ondansetron may be useful to prevent postoperative nausea and/or vomiting associated with the anesthesia administered during surgery. In some embodiments wherein the disintegrating film dosage form is used for the prevention of postoperative nausea and/or vomiting, the typical oral dosage for adults is two (2) disintegrating film dosage form each including 8 mg of ondansetron, administered about 15 minutes to about 2 hours, preferably about 30 minutes to about 1 hour, and more preferably about one hour before induction of anesthesia.

What is claimed is:

1. An orally administrable, disintegrating film dosage form comprising ondansetron, wherein the dosage form provides a mean maximum plasma concentration (Cₘₐₓ) of about 2.0 to about 4.5 μg/L per mg of ondansetron in the dosage form after oral administration of a single dosage form to human subjects in a fed state.

2. The dosage form of embodiment 1, wherein the dosage form provides a mean maximum plasma concentration (Cₘₐₓ) of about 2.2 to about 4.4 μg/L per mg of ondansetron in the dosage form after oral administration of a single dosage form to human subjects in a fed state.

3. The dosage form of embodiment 1, wherein the dosage form provides a mean maximum plasma concentration (Cₘₐₓ) of about 2.3 to about 4.3 μg/L per mg of ondansetron in the dosage form after oral administration of a single dosage form to human subjects in a fed state.

4. The dosage form of embodiment 1, wherein the Cₘₐₓ is achieved within about 3 hours of administration of the dosage form.

5. An orally administrable, disintegrating film dosage form comprising ondansetron, wherein the dosage form provides a mean maximum plasma concentration (Cₘₐₓ) of about 3.0 to about 6.9 μg/L per mg of ondansetron in the dosage form after oral administration of a single dosage form to human subjects in a fasted state.

6. The dosage form of embodiment 5, wherein the dosage form provides a mean maximum plasma concentration (Cₘₐₓ) of about 3.2 to about 6.7 μg/L per mg of ondansetron in the dosage form after oral administration of a single dosage form to human subjects in a fasted state.

7. The dosage form of embodiment 5, wherein the dosage form provides a mean maximum plasma concentration (Cₘₐₓ) of about 3.3 to about 6.5 μg/L per mg of ondansetron in the dosage form after oral administration of a single dosage form to human subjects in a fasted state.

8. The dosage form of embodiment 5, wherein the Cₘₐₓ is achieved within about 4 hours of administration of the dosage form.

9. An orally administrable, disintegrating film dosage form comprising ondansetron, wherein the dosage form provides a mean plasma concentration over 0-24 hours (AUC₀₋₂₄) of about 11.6 to about 36.0 μg·hr/L per mg of ondansetron in the dosage form after oral administration of a single dosage form to human subjects in a fed state.

10. The dosage form of embodiment 9, wherein the dosage form provides a mean plasma concentration over 0-24 hours (AUC₀₋₂₄) of about 12.9 to about 34.8 μg·hr/L per mg of ondansetron in the dosage form after oral administration of a single dosage form to human subjects in a fed state.

11. The dosage form of embodiment 9, wherein the dosage form provides a mean plasma concentration over 0-24 hours (AUC₀₋₂₄) of about 14.1 to about 33.5 μg·hr/L per mg of ondansetron in the dosage form after oral administration of a single dosage form to human subjects in a fed state.

12. An orally administrable, disintegrating film dosage form comprising ondansetron, wherein the dosage form provides a mean plasma concentration over 0-24 hours (AUC₀₋₂₄) of about 19.4 to about 44.0 μg·hr/L per mg of ondansetron in the dosage form after oral administration of a single dosage form to human subjects in a fasted state.

13. The dosage form of embodiment 12, wherein the dosage form provides a mean plasma concentration over 0-24 hours (AUC₀₋₂₄) of about 20.8 to about 42.7 μg·hr/L per mg of ondansetron in the dosage form after oral administration of a single dosage form to human subjects in a fasted state.

14. The dosage form of embodiment 12, wherein the dosage form provides a mean plasma concentration over 0-24 hours (AUC₀₋₂₄) of about 22.0 to about 41.5 μg·hr/L per mg of ondansetron in the dosage form after oral administration of a single dosage form to human subjects in a fasted state.

15. An orally administrable, disintegrating film dosage form comprising ondansetron, wherein the dosage form provides a time to reach maximum plasma concentration (Tₘₐₓ) of ondansetron of less than about 4 hours after oral administration of a single dosage form to human subjects in a fed state.

16. The dosage form of embodiment 15, wherein the dosage form provides a time to reach maximum plasma concentration (Tₘₐₓ) of ondansetron of less than about 3 hours after oral administration of a single dosage form to human subjects in a fed state.

17. An orally administrable, disintegrating film dosage form comprising ondansetron, wherein the dosage form provides a time to reach maximum plasma concentration (Tₘₐₓ) of ondansetron of less than about 3 hours after oral administration of a single dosage form to human subjects in a fasted state.

18. The dosage form of embodiment 17, wherein the dosage form provides a time to reach maximum plasma concentration (Tₘₐₓ) of ondansetron of less than about 2 hours after oral administration of a single dosage form to human subjects in a fasted state.

19. An orally administrable, disintegrating film dosage form comprising ondansetron, wherein the dosage form is configured such that when the dosage form is administered to human subjects in a fasted state with administration of water, the mean maximum plasma concentration (Cₘₐₓ) of ondansetron achieved after administration of the dosage form is within about ±10% of the mean maximum plasma concentration (Cₘₐₓ) of ondansetron achieved after administration of the dosage form when administered to human subjects in a fasted state without administration of water.

20. The dosage form of embodiment 19, wherein the dosage form is configured such that when the dosage form is administered to human subjects in a fasted state with administration of water, the mean maximum plasma concentration (Cₘₐₓ) of ondansetron achieved after administration of the dosage form is within about ±8% of the mean maximum plasma concentration (Cₘₐₓ) of ondansetron achieved after administration of the dosage form when administered to human subjects in a fasted state without administration of water.

21. The dosage form of embodiment 19, wherein the dosage form is configured such that when the dosage form is administered to human subjects in a fasted state with administration of water, the mean maximum plasma concentration (Cₘₐₓ) of ondansetron achieved after administration of the dosage form is within about ±5% of the mean maximum plasma concentration (Cₘₐₓ) of ondansetron achieved after administration of the dosage form when administered to human subjects in a fasted state without administration of water.

22. An orally administrable, disintegrating film dosage form comprising ondansetron, wherein the dosage form is configured such that when the dosage form is administered to human subjects in a fasted state with administration of water, the mean plasma concentration over 0-24 hours (AUC₀₋₂₄) of ondansetron achieved after administration of the dosage form is within about ±10% of the mean plasma concentration over 0-24 hours (AUC₀₋₂₄) of ondansetron achieved after administration of the dosage form when administered to human subjects in a fasted state without administration of water.

23. The dosage form of embodiment 22, wherein the dosage form is configured such that when the dosage form is administered to human subjects in a fasted state with administration of water, the mean plasma concentration over 0-24 hours (AUC₀₋₂₄) of ondansetron achieved after administration of the dosage form is within about ±5% of the mean plasma concentration over 0-24 hours (AUC₀₋₂₄) of ondansetron achieved after administration of the dosage form when administered to human subjects in a fasted state without administration of water.

24. The dosage form of embodiment 22, wherein the dosage form is configured such that when the dosage form is administered to human subjects in a fasted state with administration of water, the mean plasma concentration over 0-24 hours (AUC₀₋₂₄) of ondansetron achieved after administration of the dosage form is within about ±1% of the mean plasma concentration over 0-24 hours (AUC₀₋₂₄) of ondansetron achieved after administration of the dosage form when administered to human subjects in a fasted state without administration of water.

25. An orally administrable, disintegrating film dosage form comprising ondansetron, wherein the dosage form is configured such that when the dosage form is administered to human subjects in a fasted state with administration of water, the time to reach maximum plasma concentration (Tₘₐₓ) of ondansetron achieved after administration of the dosage form is within about ±20% of the time to reach maximum plasma concentration (Tₘₐₓ) of ondansetron achieved after administration of the dosage form when administered to human subjects in a fasted state without administration of water.

26. The dosage form of embodiment 25, wherein the dosage form is configured such that when the dosage form is administered to human subjects in a fasted state with administration of water, the time to reach maximum plasma concentration (Tₘₐₓ) of ondansetron achieved after administration of the dosage form is within about ±18% of the time to reach maximum plasma concentration (Tₘₐₓ) of ondansetron achieved after administration of the dosage form when administered to human subjects in a fasted state without administration of water.

27. The dosage form of embodiment 25, wherein the dosage form is configured such that when the dosage form is administered to human subjects in a fasted state with administration of water, the time to reach maximum plasma concentration (Tₘₐₓ) of ondansetron achieved after administration of the dosage form is within about ±15% of the time to reach maximum plasma concentration (Tₘₐₓ) of ondansetron achieved after administration of the dosage form when administered to human subjects in a fasted state without administration of water.

28. A method of treating, preventing, and/or reducing the occurrence of nausea and/or vomiting, comprising administering the dosage form of any of claims 1-27.

29. The method of embodiment 28, wherein the nausea and/or vomiting is associated with chemotherapy.

30. The method of embodiment 29, wherein the chemotherapy is a highly emetogenic cancer chemotherapy or a moderately emetogenic cancer chemotherapy.

31. The method of embodiment 28, wherein the nausea and/or vomiting is associated with radiotherapy.

32. The method of embodiment 31, wherein the radiotherapy is selected from the group consisting of: total body irradiation, single high-dose fraction radiotherapy to the abdomen, and daily fractionated radiotherapy to the abdomen.

33. The method of embodiment 28, wherein the nausea and/or vomiting is postoperative nausea and/or vomiting.

34. An orally administrable, disintegrating film dosage form comprising ondansetron and one or more film-forming polymers.

35. The dosage form of claim 34, wherein the film forming-polymer is selected from the group consisting of: water soluble polymers, water insoluble polymers, and a combination of one or more water soluble polymers and/or water insoluble polymers.

36. The dosage form of embodiment 34, wherein the dosage form comprises at least one cellulose polymer or cellulosic derivative polymer.

37. The dosage form of embodiment 35, wherein the cellulose polymer or cellulosic derivative polymer is selected from the group consisting of: methylcellulose, hydroxymethylcellulose, hydroxyethylcellulose, hydroxypropylcellulose, hydroxyethyl methylcellulose, hydroxypropylmethylcellulose, hydroxybutylmethylcellulose, cellulose acetate phthalate, carboxymethylcellulose and their alkali metal salts.

38. The dosage form of embodiment 34, wherein the dosage form further comprises at least one synthetic polymer.

39. The dosage form of embodiment 38, wherein the synthetic polymer is selected from the group consisting of: polyacrylic acids and polyacrylic acid esters, polyalkylene oxides, polymethacrylic acids and polymethacrylic acid esters, polyvinylacetates, polyvinylalcohols, polyvinylacetatephthalates (PVAP), polyvinylpyrrolidone (PVP), polyvinyl acetate (PVA) and polyvinyl acetate copolymers, and polycrotonic acids.

40. The dosage form of embodiment 39, wherein the synthetic polymer comprises polyethylene oxide.

41. The dosage form of embodiment 38, wherein the amount of cellulose polymers or cellulosic derivative polymers is greater than the amount of the synthetic polymers.

42. The dosage form of embodiment 38, wherein the amount of cellulose polymers or cellulosic derivative polymers are present in a weight ratio ranging from about 10:1 to about 1:10.

43. The dosage form of embodiment 38, wherein the amount of cellulose polymers or cellulosic derivative polymers are present in a weight ratio ranging from about 7:1 to about 1:7.

44. The dosage form of embodiment 38, wherein the amount of cellulose polymers or cellulosic derivative polymers are present in a weight ratio ranging from about 1:1 to about 3:1.
